(19) United States
(12) Patent Application Publication
(10) Pub. No.: US 2003/0023233 A1
(43) Pub. Date: Jan. 30, 2003

(54) Technique for removal of material to achieve a desired shape with a laser

(76) Inventors: Michael J. Smith, Orlando, FL (US); Lance R. Marrou, Orlando, FL (US)

Correspondence Address: MANELLI DENISON & SELTER PLLC, 7th Floor, 2000 M Street, N.W., Washington, DC, 20036-3307, US

(21) Appl. No.: 10/207,127

(22) Filed: Jul. 30, 2002

Related U.S. Application Data
(60) Provisional application No. 60/308,131, filed on Jul. 30, 2001.

(51) Int. Cl. A61B 18/20

(52) U.S. Cl. 606/5

(57) ABSTRACT

Methods are provided for material sculpting with a device which functions similar to a laser on human cornea or plastic that reduces surface roughness and computational overhead. These methods are based upon teachings of volumetric ablation based upon layer slices of the volume. The first method demonstrates how to apply the slice based sculpting to a regularly spaced sampling grid using stochastic perturbing from the grid points. The second method demonstrates how to apply a directly volumetric sculpting approach where a discrete estimate of the average beam size and shape is made and applied directly to the volume to ablate until all of the required volume is removed.

This application is based on and claims priority from U.S. Provisional Application No. 60/308,131 filed on Jul. 30, 2001, the entirety of which is expressly incorporated herein by reference.

### BACKGROUND OF THE INVENTION

1. Field of the Invention

The present invention relates to a method for removing a specific amount of material from specific locations on a surface such as plastic, human corneal tissue or other ablative materials. This invention applies to the positioning of laser pulses in refractive surgery, shaped human tissue removal, and shaped removal of other materials that can be ablated.

2. Background

For lasers or sculpting systems that use a material vaporization or photo-ablation system, the set of possible shapes available for a large fixed beam and a mask system is limited to the combination of mask shapes. For refractive laser systems, the use of an iris mask limits shapes to primarily circularly symmetric volumes. For smaller moveable or positioning beam laser systems, the set of available shapes that can be generated is only limited by the size of the beam and the irregularity of the material removed with each shot. For small beam systems, a technique which views a volume to sculpt as a set of layers and uses an algorithm to remove material inside the shape at the given volume slice can achieve reasonable results. If a regular grid is used to sample the volume to ablate, a problem can occur where the pulse irregularity stacks up and magnifies the apparent error on the removed volume. Lin U.S. Pat. No. RE 37,504 teaches this method and also shows some possible methods for reducing the effect. Alternative methods of ablation are taught in the present invention.

### SUMMARY OF THE INVENTION

The object of the invention is to provide alternative sculpting methods that provide more accurate results or provide a higher degree of precision in the resulting volume sculpted. The first method procedure shown in FIG. 1 is also graphically depicted in a plan view in FIG. 3. This method builds on previous work that breaks volume sculpting down into layers and removes material according to each layer slice. The variation this method makes is the selection of locations to make up the locations to remove material. The primary feature of this method is that is makes use of a stochastic perturbation of the material removal location to reduce the aliasing effects that become apparent if a regularly spaced grid is used for each layer.

The second method approaches the problem of volumetric sculpting by choosing to discretize the sculpting beam and the volume size, shape, and depth. After these items have been discretized, the process involves removing tissue at stochastically selected locations, based on a pulse restriction function, where the material left to remove is primarily required to meet the original volume shape goal. This method's procedure flow is demonstrated in FIG. 2 and can be seen graphically in a plan view in FIG. 4 or in a cross-section in FIG. 5.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, in which:

FIG. 1 is a flow chart showing a technique for performing volume sculpting using a volume slice method with stochastic perturbing of grid points. A graphical depiction of this method is given in FIG. 3.

FIG. 2 is a flow chart showing the technique for volume sculpting using the volume discretization approach with restricted stochastic placement of pulses. A graphical depiction of this method is given in FIG. 4 and FIG. 5.

FIG. 3 is an exemplary plan view of the volume slice method with stochastic perturbing of grid points, in accordance with the present invention.

FIG. 4 is an exemplary plan view of the volume discretization approach with restricted stochastic placement of pulses, in accordance with the present invention.

FIG. 5 is an exemplary cross-section of the volume discretization approach with restricted stochastic placement of pulses, in accordance with the present invention.

### DETAILED DESCRIPTION OF ILLUSTRATIVE EMBODIMENTS

Volume Sculpting Using a Volume Slice Method with Stochastic Perturbing

In the method, the ablation profile is divided horizontally into slices that are a known or predetermined thickness. These slices, called layers, are then checked for points within the boundary of the ablation profile using a regularly spaced grid so that neighboring pulses can overlap.

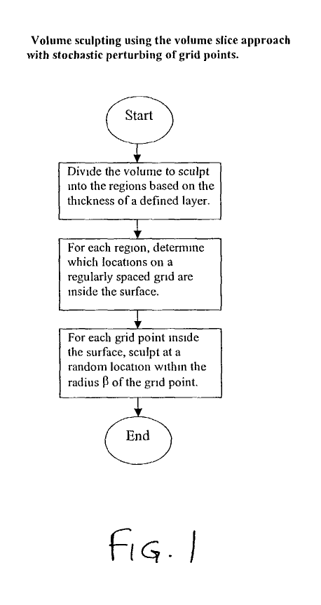

In the prior art, the pulses inside the boundary of the ablation profile are then ablated to remove the material for the layer. The present invention improves upon the prior art by eliminating most of the aliasing from the regularly spaced grids on each layer. As outlined in FIG. 1, the present invention first determines the regions defined by the ablation profile for each slice. Then, each region is compared to a regular grid and chosen for inclusion in the ablation. This is similar to the prior art. For the actual ablation, however, each chosen point will be repositioned by up to a distance β (referred to as radius β in the figure) from the ideal point to prevent direct beam stack up on a single location.

The distance each chosen point moves will be stochastically chosen within the radius β so consecutive layers in the same region will not offset the actual ablation point in the same location. The choice of this offset can use stochastic perturbation or methods that create a similar result such as functions that evenly distribute pulses for each regular grid point across layers. A reasonable first choice for β is ¼ to ½ of the size of the ablation beam. With this technique, the computational cost to decide the location to ablate is very low and can produce a result that does not suffer from aliasing errors as the basic method. The stochastic sampling function is not further detailed in the preferred embodiment, but exemplary functions are readily available.

Volume Sculpting Using a Volume Discretization Approach with Restricted Stochastic Placement of Pulses

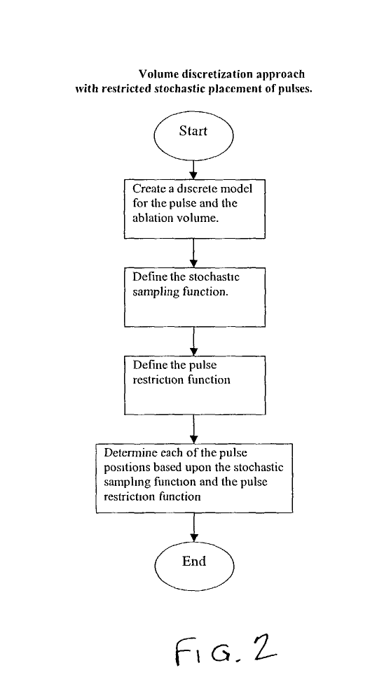

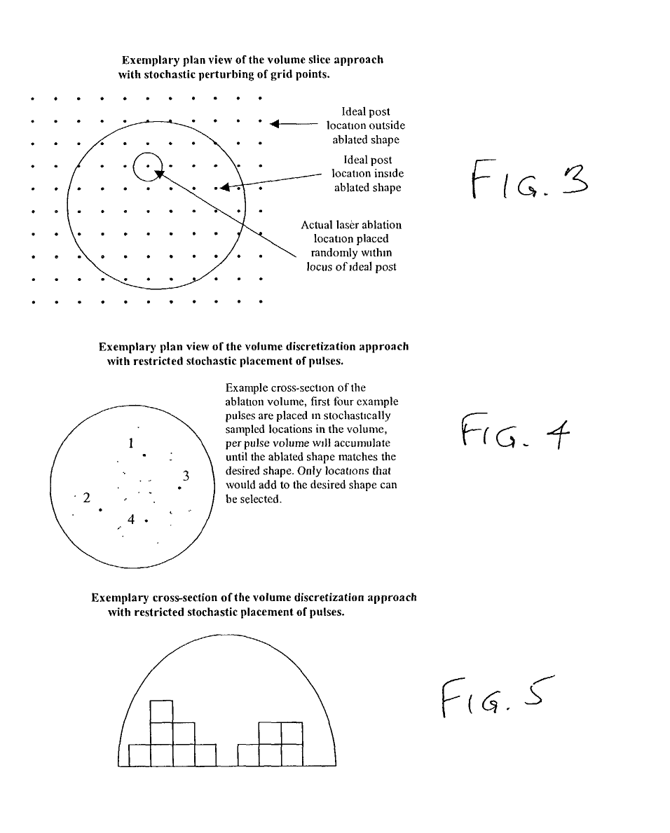

The approach of the present invention focuses on setting up a discrete system to express the size and shape of the laser beam and target ablation profile volume. Then, a pulse restriction function is defined to restrict the placement of shots within the volume so that the volume is generated correctly according to the design of the device. This approach is expressed in terms of using a laser to photo-ablate material from a surface but can be directly applied to similar material removal processes that target a specific volumetric shape. This approach is outlined in FIG. 2.

An exemplary discretization of the laser pulse divides the beam into a regularly spaced grid of 5×5. The same grid spacing and scale would then also be used for the ablation profile, but only in the horizontal direction. The height of the volume does not necessarily need to be discretized as in the previous approach outlined above. Given an exemplary pulse width of 1.0 mm and the exemplary grid above, each grid size is 0.2 mm.

Then, the first pulse location would be stochastically chosen in the horizontal plane. The first place will likely not be restricted, except based upon the horizontal area of the volume. Each subsequent location would also be chosen stochastically, restricted by the pulse restriction function. The pulse restriction function must be defined for each system and usually depends upon the characteristics of that system. One example for the pulse restriction function is to disallow previous locations until a certain height or number of stacked pulses is reached in that location. Another would be to target (create a higher probability for) the current highest point of the volume that is left to be ablated. In accordance with the present invention, any number of pulse restriction functions may be defined, as necessary for different systems using different beam widths, pulse volumes, fluence, or surgical algorithms.

Exemplary pulse locations are shown in the exemplary plan view in FIG. 4. Each pulse would subtract from the volume, given the predefined discretization of the pulse and the volume to ablate. Only locations where the ablation will favorably add to creating the targeted shape will be allowed. This buildup in the volume that was ablated and the remaining volume to ablate is graphically depicted in FIG. 5. As can be seen in the exemplary cross-section view, the pulses can buildup on each other in mostly random locations, depending on the pulse restriction function as described above.

Additional modifications to the stochastic function in this approach could be provided that would help eliminate buildup of pulses in one location or otherwise improve the ablation method for the given application. One example of this would be to discretize the volume in the vertical domain, thus creating the layers similar to the prior approach and prior art. Another example would be to limit the number of pulses that may be stacked on top of each other to two or three until such a time as the entire slice has been ablated. As suggested above, this might be better implemented in the pulse restriction function rather than a change to the stochastic sampling function. A final example would be to restrict the location of the pulses to limit the gradient of the shape of the built-up pulses. As shown in FIG. 5, no pulses are stacked higher than 3. Any additional, randomly placed pulses would be restricted to an area with less than 3 current pulses. The example does not illustrate overlapping pulses, although such an implementation is possibly under the present invention.

These aforementioned methods are not limited to refractive surgical devices, but may rather be used on any volume sculpting device that functions in a similar manner.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

### Definitions

Aliasing—The appearance of jagged distortions in curves and diagonal lines in computer graphics because the resolution is limited or diminished. Applied to a sculpted surface, this would appear as pits or irregularities that are gross departures from the intended target ablation profile.

Radius β—A term used in this paper to denote the distance to actually sculpt (i.e. fire a laser beam) from the ideal location of energy delivery. The actual value for the distance is normally ¼ to ½ the size of the beam. This value may need to be changed experimentally upon need.

Refractive Surgery—Surgery performed to bring about a refractive change in the human vision system to account for vision problems that require glasses to achieve normal vision or correct corneal blindness not correctable by glasses.

Volume Sculpting—Removing material to achieve a desired shape using a subtractive or additive process.

What is claimed is:

1. A method of volume sculpting, said method comprising:
dividing a desired volume of tissue to be ablated into a plurality of slices each of a known thickness;
determining an overlaid grid that is smaller than a size of an ablating laser beam spot;
identifying grid points inside said desired volume of tissue to sculpt, said grid points being produced by overlaying said grid over each of said plurality of slices; and
firing said ablating laser beam spot from a distance β from each of said identified grid points inside said desired volume of tissue to be ablated.

2. The method of volume sculpting according to claim 1, wherein:
said known thickness of each of said plurality of slices is approximately equal.

3. The method of volume sculpting according to claim 1, wherein:
said tissue is corneal tissue.

4. The method of volume sculpting according to claim 1, wherein:
said method of volume sculpting is comprised in corneal refractive surgery.

5. A method of volume sculpting, said method comprising:
creating a discrete model of a volume of tissue to be ablated;
defining a stochastic sampling function;
defining a pulse restriction function; and
pulsing an ablating laser beam in locations of a volume to be ablated based on both said defined stochastic sampling function and said pulse restriction function.

6. The method of volume sculpting according to claim 5, wherein:
said tissue to be ablated is corneal tissue.

7. Apparatus for volume sculpting, comprising:
means for dividing a desired volume of tissue to be ablated into a plurality of slices each of a known thickness;
means for determining an overlaid grid that is smaller than a size of an ablating laser beam spot;
means for identifying grid points inside said desired volume of tissue to sculpt, said grid points being produced by overlaying said grid over each of said plurality of slices; and
means for firing said ablating laser beam spot from a distance β from each of said identified grid points inside said desired volume of tissue to be ablated.

8. The apparatus for volume sculpting according to claim 7, wherein:
said known thickness of each of said plurality of slices is approximately equal.

9. The apparatus for volume sculpting according to claim 7, wherein:
said apparatus is adapted to sculpt corneal tissue.

10. The apparatus for volume sculpting according to claim 7, wherein:
said apparatus is comprised in corneal refractive surgery.

11. Apparatus for volume sculpting, comprising:
means for creating a discrete model of a volume of tissue to be ablated;
means for defining a stochastic sampling function;
means for defining a pulse restriction function; and
means for pulsing an ablating laser beam in locations of a volume to be ablated based on both said defined stochastic sampling function and said pulse restriction function.

12. The apparatus for volume sculpting according to claim 11, wherein:
said apparatus is adapted to ablate corneal tissue.
